(19) United States
(12) Patent Application Publication
(10) Pub. No.: US 2024/0115676 A1
(43) Pub. Date: Apr. 11, 2024

(54) SILICIFIED TUMOR CELL COMPOSITIONS AND METHODS

(71) Applicants: Rita E. Serda, Albuquerque, NM (US); Jimin Guo, Albuquerque, NM (US); UNM Rainforest Innovations, Albuquerque, NM (US)

(72) Inventors: JIMIN GUO, Albuquerque, NM (US); RITA E. SERDA, Albuquerque, NM (US)

(73) Assignee: UNM Rainforest Innovations, Albuquerque, NM (US)

(21) Appl. No.: 18/267,878

(22) PCT Filed: Dec. 14, 2021
(86) PCT No.: PCT/US2021/063386

§ 371 Date: Jun. 16, 2023

Related U.S. Application Data
(60) Provisional application No. 63/126,110, filed on Dec. 16, 2020.

(51) Int. Cl. A61K 39/00; A61P 35/00; A61P 37/04

(52) U.S. Cl. CPC A61K 39/0011; A61P 35/00; A61P 37/04; A61K 2039/5152; A61K 2039/55561

(57) ABSTRACT

In one aspect, a method generally includes obtaining a dried silicified cell that has been stored for at least 24 hours without cryopreservation and rehydrating the dried silicified cell in a pharmaceutically acceptable carrier. The method can further include surface modifying the silicified cell with at least one immunogenic molecule. The method can further include administering the rehydrated silicified cell to a subject. In some embodiment, the dried silicified cell has been stored for at least 14 days without cryopreservation. In another aspect, a method of treating a tumor in a subject generally includes administering to the subject a chemotherapeutic agent effective to treat the tumor and administering to the subject a silicified cell vaccine effective to treat the tumor.

### CROSS-REFERENCE TO RELATED APPLICATION

This application claims the benefit of U.S. Provisional Patent Application No. 63/126,110, filed Dec. 16, 2020, which is incorporated herein by reference in its entirety.

### GOVERNMENT FUNDING

This invention was made with government support under CA118100 awarded by the National Institutes of Health. The government has certain rights in the invention.

### SUMMARY

This disclosure describes, in one aspect, a method that generally includes obtaining a dried silicified cell that has been stored for at least 24 hours without cryopreservation and rehydrating the dried silicified cell in a pharmaceutically acceptable carrier.

In some embodiments, the method further includes surface modifying the silicified cell with at least one immunogenic molecule.

In some embodiments, the method further includes administering the rehydrated silicified cell to a subject.

In some embodiments, the dried silicified cell has been stored for at least 14 days without cryopreservation.

In some embodiments, the silicified cell is a silicified tumor cell.

In some embodiments, the silicified cell is derived from a tumor cell line.

In some embodiments, the silicified cell is a de-differentiated cell.

In another aspect, this disclosure describes a method of treating a tumor in a subject. Generally, the method includes administering to the subject a tumor therapy effective to treat the tumor and administering to the subject a silicified cell vaccine effective to treat the tumor.

In some embodiments, the tumor therapy comprises a chemotherapeutic agent.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Characterization of silicified cancer cells. (A) Cryo-silicification and adsorption of PAMPs to cancer cells. (B) Confirmation of Si content in silicified BR5-Akt tumor cells using SEM and energy dispersive X-ray analysis of carbon (C), oxygen (O), and silicon (Si). (C) ICP-OES analysis of Si content in BR5-Akt cells silicified using either 10 mM or 100 mM silicic acid solution for 24 hours at 20° C. or −80° C.; and Si content in 10 mM cryo-silicified cells before and after 72 hours in simulated endosomal fluid (n=3/condition). (D) Zeta potential analysis of silicified cells with different surface modifications (n=6/group).

FIG. 2. Characterization of silicified cancer cells. (A) Pie charts showing Si-PEI-CpG-MPL cell composition by mass ratio. (B) Cell-Glo proliferation assay of live or silicified BR5-Akt cells, with and without a 10-minute immersion in 0.2 mg/mL PEI measured at 24 hours, 48 hours, and 72 hours (n=3/condition). (C) Tumor burden over time based on IVIS bioluminescence of FVB mice IP injected on Day 0 with either live (cancer challenged) or silicified (vaccine only) BR5-Akt-Luc2 cells (n=3/group). *p<0.05, **p<0.01, ***p<0.001. (D) Flow cytometry scatter dot plots of live or silicified BR5-Akt cells showing change in size (FSC) with silicification and histogram of cells before or after staining with propidium iodide (PI) to demonstrate that silicified cells are nonviable (n=3/group).

FIG. 3. Surface functionalization enhances dendritic cell uptake and activation in vitro. (A) 2D and surface-rendered 3D fluorescent confocal micrographs showing internalization and intracellular location of silicified tumor cells following a one-hour incubation with GM-CSF-matured BMDC. Actin fluorescence is shown at three threshold levels for inside and surface views. The white arrow points to active phagocytosis. (B) 2D and surface-rendered 3D fluorescent confocal micrographs showing internalization and intracellular location of silicified tumor cells following a one-hour incubation with GM-CSF-matured BMDC. Tumor cells were pre-incubated with fluorescent nanoparticles prior to silicification to distinguish vaccine cells from dendritic cells.

FIG. 4. Surface functionalization enhances dendritic cell uptake and activation in vitro. (A) Flow cytometry analysis of dendritic cell uptake of silicified BR5-Akt cells presenting no TLR ligands (Si); PEI; or PEI, CpG and MPL (n=8/group). (B) Flow cytometry analysis of MHC I presentation of tumor antigen (SIINFEKL-H2Kb) on dendritic cells 72 hours after addition of ID8ova vaccine cells or control irradiated ID8ova cells (n=3/group). ***p<0.001; ****p<0.0001.

FIG. 5. Treatment with silicified cells induces a protective immune response in vivo. Tumor engraftment was evaluated in FVB mice challenged with BR5-Akt-Luc2 cells. Kaplan-Meier survival curves and tumor burden (graphically and as IVIS images). Antigen specificity was tested by intraperitoneal injection with vaccine cells or no antigen control mesoporous silica nanoparticles (MSN) presenting PEI, CpG, and MPL 21 days prior to tumor challenge (n=4/group). **p<0.01.

FIG. 6. Treatment with silicified cells induces a protective immune response in vivo. The ability of intraperitoneal (IP) or subcutaneous (SC) treatment with Si-PEI-CpG-MPL cells to protect against intraperitoneal tumor challenge was evaluated (n=4/group). *p0.05; **p<0.01.

FIG. 7. Treatment with silicified cells induces a protective immune response in vivo. The existence of local memory T cells was evaluated by adoptive transfer (AT) of CD8-enriched peritoneal cells from vaccinated mice to naïve mice, with intraperitoneal tumor challenge 24 hours post vaccination with BR5-Akt-Luc2 cells (n=10/group). **p<0.01; ***p<0.001; ****p<0.0001.

FIG. 8. Therapeutic benefit of vaccination is associated with significant changes in tumor-associated lymphocytes. (A) 3D IVIS bioluminescent images of tumor burden on Day 2, Day 4, Day 8, and Day 16 post tumor challenge (n=3/group). (B) Luminex IL-4 cytokine and flow cytometry evaluation of peritoneal T cell activation status at the various stages of tumor progression (n=3/group).

FIG. 9. Flow cytometry was used to define changes in peritoneal T cell type and activation status following vaccination (n=4/group). (A) Female FVB mice were injected intraperitoneally with 2×10⁵ BR5-Akt-Luc2 cancer cells on Day 0, vaccinated on Days 4 and Day 11 with 3×10⁶ BR5-Akt vaccine cells (Si Vac) or vehicle PBS (no Tx), and peritoneal fluid/wash was collected for analysis on Day 25. (B) Percent of intraperitoneal CD4⁺ and CD8⁺ T cells with naïve (CD44⁻CD62L(high)), central memory (CD44⁺CD62L(high)), and effector memory (CD44⁺CD62L(low)) phenotypes. (C) Number of intraperitoneal CD4⁺ and CD8⁺ T cells with naïve (CD44⁻CD62L(high)), central memory (CD44⁺CD62L(high)), and effector memory (CD44⁺CD62L(low)) phenotypes.

FIG. 10. Therapeutic benefit of vaccination is associated with significant changes in tumor-associated lymphocytes. (A) Percent of CD4⁺ and CD8⁺ cells expressing IFNγ after re-stimulation with PMA/ionomycin, and percent of intraperitoneal CD4⁺ cells expressing regulatory T cell markers FoxP3 and CTLA4. (B) Flow cytometry analysis of Th response using peritoneal cells or fluid from mice (n=8/group) receiving dehydrated vaccine or no treatment (no Tx; Day 27). (C) Luminex cytokine analysis of Th response using peritoneal cells or fluid from mice (n=8/group) receiving dehydrated vaccine or no treatment (no Tx; Day 27).*p<0.05, **p<0.01, ***p<0.001, ****p<0.0001.

FIG. 11. Therapeutic vaccination clears established tumors and promotes a Th1 response. Diagram of treatment schedule and tumor burden (IVIS images) in FVB mice intraperitoneally injected with tumor cells followed by treatment with free adjuvant (free) or vaccination with silicified (Si) or irradiated (IR) cancer cells, with and without adjuvant.

FIG. 12. Therapeutic vaccination clears established tumors and promotes a Th1 response. Tumor burden (photons/sec; n=10/group) and Kaplan-Meier survival curves (n=10/group).

FIG. 13. Therapeutic vaccination clears established tumors and promotes a Th1 response. (A) Photographs of irradiated (IR) or silicified (Si) cancer cells following dehydration (dry) and suspension in PBS (rehydrate). Bright field micrographs of fixed or Si cells pre-/post-dehydration and suspension in PBS. (B) Clearance of established bioluminescent tumors in FVB mice receiving dehydrated vaccine intraperitoneally on Day 4 and Day 11 compared to no treatment (control) or fresh (non-dehydrated) vaccine (n=8/group). *p<0.05, **p<0.01, ***p<0.001, ****p<0.0001.

FIG. 14. Combination cisplatin and vaccine therapy clears tumors and enhances survival in mice. IVIS images in FVB mice intraperitoneally injected with tumor cells followed by treatment with free adjuvant (no Tx), treatment with cisplatin alone, treatment with vaccine alone, or treatment with vaccine and cisplatin.

FIG. 15. Timeline plus tumor burden and Kaplan-Meier survival curves of tumor-bearing FVB mice treated intraperitoneally with cisplatin (cis) on Day 9, vaccine (vac) on Day 10, or cisplatin and vaccine (cis+vac) on Days 9 and Day 10 post intraperitoneal tumor injection (n=10/group). *p<0.05, **p<0.01, ***p<0.001, ****p<0.0001.

FIG. 16. Effective vaccine can be developed from ascites. (A) Treatment schedule and diagram showing vaccine preparation using ascites from a FVB mouse with late stage BR5-Akt ovarian cancer. (B) Flow cytometry analysis of EpCAM⁺ cells in mouse or human ascites before and after filtration enrichment.

FIG. 17. Effective vaccine can be developed from ascites. Tumor burden and Kaplan-Meier survival curves (n=4/group) of mice treated as indicated in FIG. 16A.

FIG. 18. Effective vaccine can be developed from ascites. Tumor burden in mice intraperitoneally vaccinated on Day 4 and Day 11 with cell line (BR5 vac) or ascites vaccines (n=4/group); IVIS Spectrum bioluminescent images. To test immunological memory, vaccinated mice were re-challenged with BR5-Akt-Luc2 tumor cells on Days 36. **p<0.01, ****p<0.0001.

FIG. 19. Enhanced vaccine-stimulated activation of dendritic cells by treatment of cancer cells with doxorubicin. Surface expression of CD40, evaluated using fluorescent antibodies and flow cytometry, was used as a metric for dendritic cell activation. Dendritic cells (CD11c⁺) were incubated with PBS [no Treatment (no Tx)], silicified PEI-CpG-MPL tumor cells (vac), supernatant from live cancer cells treated with 5 ng/mL doxorubicin (dox 5), supernatant from live cancer cells treated with 50 ng/mL doxorubicin (dox 50), or a combination. Combined treatment of dendritic cells with vaccine and chemotherapy-treated cell supernatant resulted in significantly greater activation than with vaccine or supernatant alone.

FIG. 20. Vaccination with biomineralized ovarian cancer cells eliminates established tumors. Female FVB mice (6 weeks old) with established tumors were treated intraperitoneally with silicified BR5-Akt vaccine cells (3×10⁶ vaccine cells injected on Day 4 and Day 11 post intraperitoneal tumor injection). (A) Timeline of study. Mice were challenged on Day 0 and Day 104 with BR5-Akt cancer cells intraperitoneally. (B) Tumor burden over time for untreated and vaccinated mice. (C) Survival over time for untreated and vaccinated mice. (D) IVIS images show that vaccination of mice with existing tumors leads to complete tumor cell elimination. Following a rechallenge with cancer at Day 104, mice again eliminate cancer, supporting the existence of memory immune cells and long-term cancer-free survival.

### DETAILED DESCRIPTION OF ILLUSTRATIVE EMBODIMENTS

This disclosure describes a vaccine that includes silicified cancer cells presenting TLR ligands. The vaccine biosilicification process allows dry storage of the vaccine. The vaccine also works synergistically with other therapies (e.g., chemotherapy).

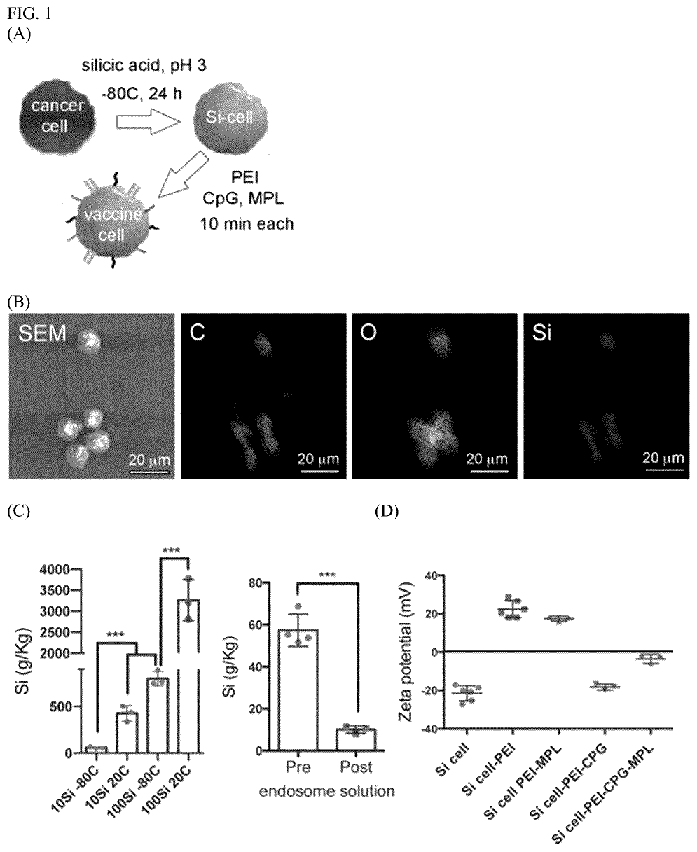

Tumor cell silicification establishes a modular platform for vaccine development. Ex vivo silicification of cancer cells as a method of fixation preserves cell integrity, preserves biofunctionality of proteinaceous components, and enables surface functionalization of the silicified cell (FIG. 1A). Cryo-silicification is technically simple and can be extended to any cell type, enabling personalized vaccine design. Methods of silicifying cells are described in U.S. Patent Application Publication No. US 2020/0276286 A1 and International Publication No. WO 2020/185449 A1.

Scanning electron microscopy (SEM) and energy dispersive spectroscopy (EDS) analysis of silicified murine ovarian cancer cells confirmed the presence of organic matter (C and O) and elemental Si (FIG. 1). Inductively coupled plasma-optical emission spectroscopy (ICP-OES) analysis of silicified cells demonstrated that this approach resulted in significantly lower Si content compared to silicification under higher silicic acid concentrations or temperature (FIG. 1C).

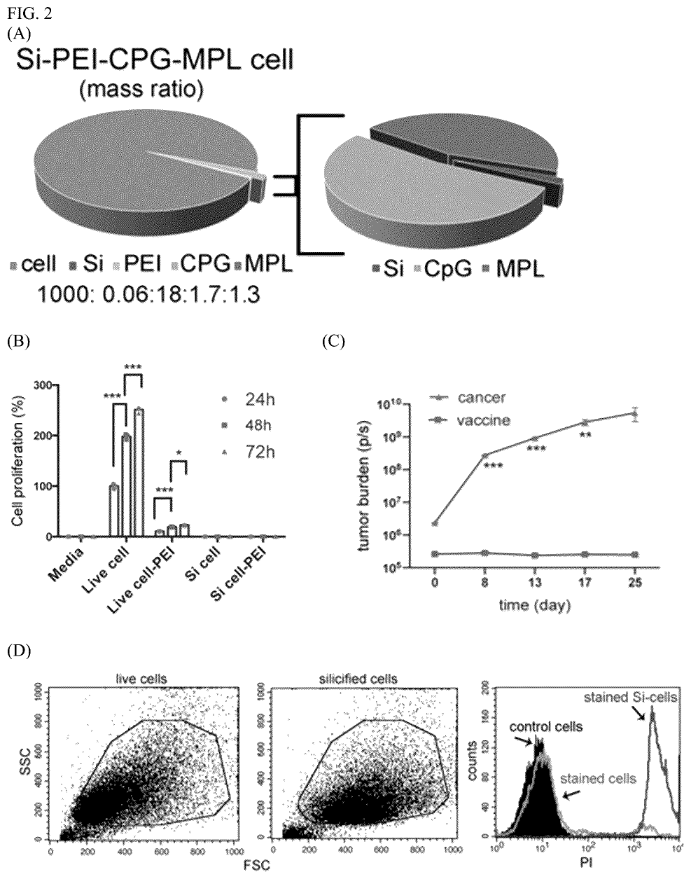

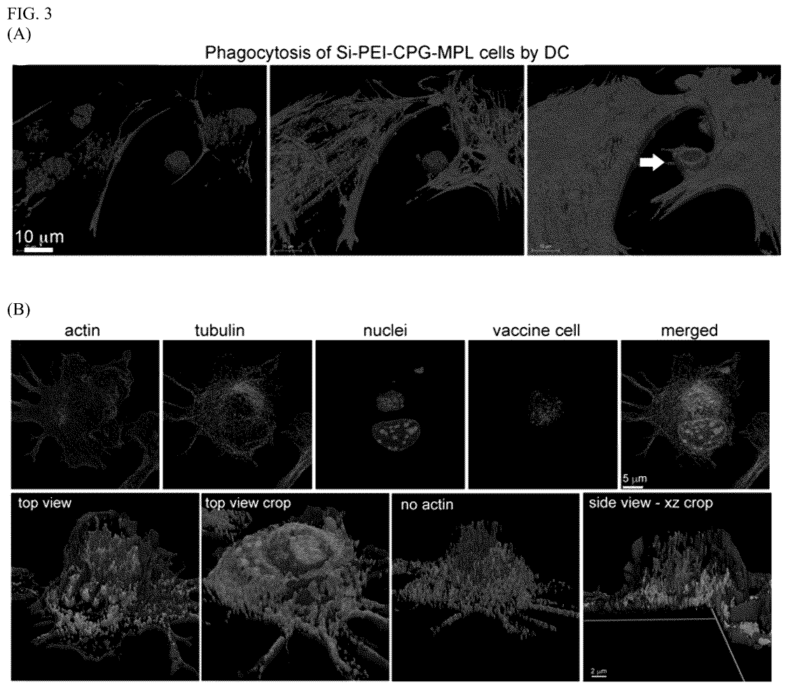

Surface modification of silicified (Si)-cells enables functionalization of silicified cells. One can functionalize silicified cells by binding one or more adjuvants or other immunomodulatory compounds to the surface of silicified cells. The native chemistry of the silicified cell surface is dominated by hydroxyl (silanol ≡Si—OH) groups. At physiological pH, the silanol groups are largely dehydroxylated creating an anionic (≡Si—O⁻) surface that adsorbs cationic molecules and polymers that, in turn, can adsorb and retain anionic ligands. Adsorption of polyethyleneimine (PEI), polylysine (PL) or chitosan (Chit) on the silicified (Si) cell surface each reversed the negative Si-cell surface/zeta potential. PEI is an organic cationic polymer that may be a ligand of TLR-4 and/or TLR-5. This, and other properties of PEI, led to PEI being selected as a model cationic surface modifier for vaccine development. CpG and monophosphoryl lipid A (MPL) were selected as model TLR agonists. Stepwise changes in zeta potential following surface modification with PEI, CpG and MPL are shown in FIG. 1D. A vaccine dose of 3×10⁶ cells contains approximately 5 μg CpG, 4 μg MPL, 54 μg PEI and 0.2 μg Si, with a mass ratio of 1000:0.06:18:1.7:1.3 for cells:Si:PEI:CpG:MPL (assuming a mass of 1 mg for 1×10⁶; FIG. 2A).

Silicified cells are safe for use in vivo. To evaluate the safety of silicified tumor cells for cancer treatment, their viability was evaluated in vitro and in vivo. During vaccine preparation, cancer cells were exposed to an acidic, hypotonic solution, followed by freezing at −80° C., and then surface modification that included incubation in 0.2 mg/ml PEI for 10 minutes. Using an absence of ATP production as an in vitro measure of metabolic activity, these processing methods resulted in complete cell death (FIG. 2B). PEI is a cationic macromolecule, and both branched and linear free PEI can compromise membrane integrity and initiate cell apoptosis. While nanoparticles with large surface areas can be cytotoxic in vitro under typical cell culture conditions, larger entities coated with PEI do not induce toxicity. In addition, in the presence of physiologically relevant levels of serum, the cationic surface charge of PEI-coated particles is masked with serum proteins, mitigating the biological effect. As an additional assessment for viability, cellular uptake of propidium iodide (PI) was evaluated by flow cytometry. Notably, scatter dot plots of live or silicified tumor cells supported retention of cell structure following silicification. In these experiments, all silicified cells displayed intracellular PI staining (FIG. 2D), confirming that silicified cells are not viable. Finally, to ensure that these cells could not establish tumors in vivo, luciferase positive silicified cells were injected intraperitoneally into mice and tumor growth was assessed for 25 days. None of these mice developed any bioluminescent evidence of tumor viability (FIG. 2C). Additionally, mice injected with silicified tumor cells by subcutaneous administration lacked evidence of any tumor at the injection site by histologic assessment.

Surface modification of Si-cells with TLR agonists enhances dendritic cell uptake and activation. Engagement of TLR4 and TLR9 on antigen-presenting cells (APC) promotes antigen internalization, cytokine secretion, and expression of costimulatory molecules and major histocompatibility complex (MHC). Co-culture experiments were performed ex vivo with bone marrow derived dendritic cells to test whether surface modification with CpG and MPL, as model TLR agonist surface modifications, promoted silicified tumor cell uptake and processing by antigen presenting cells.

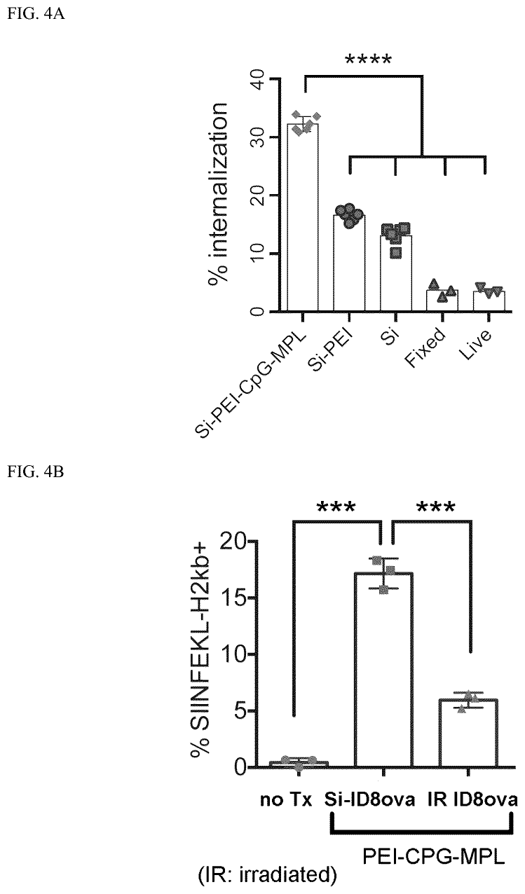

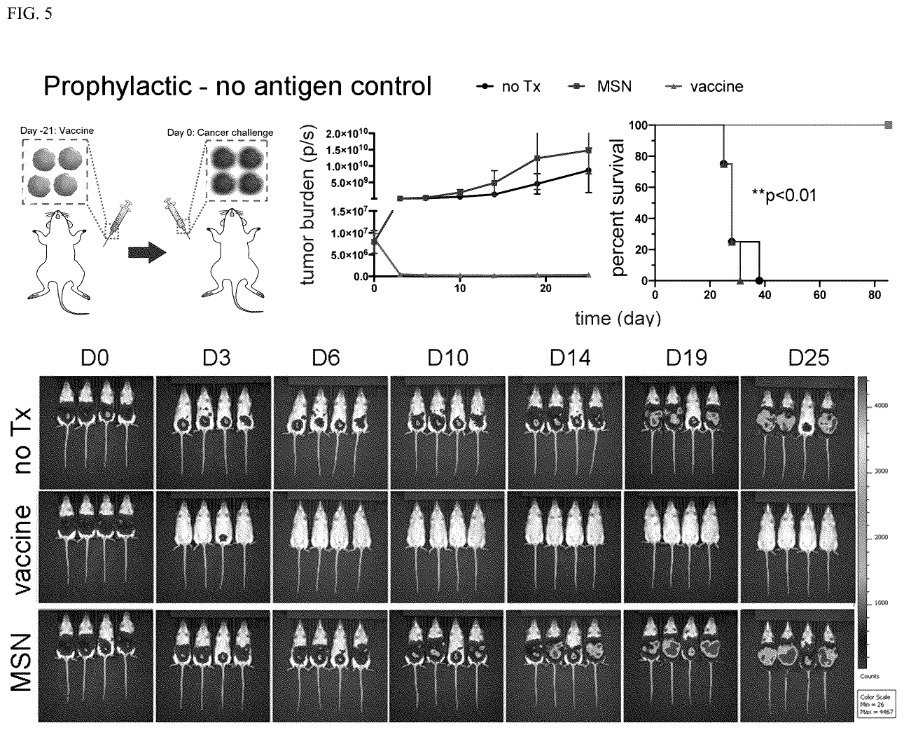

Confocal microscopy and flow cytometry confirmed that silicified tumor cells are engulfed by dendritic cells in vitro (FIG. 3). Three-dimensional confocal images of actin-labeled (red: rhodamine phalloidin; blue: DAPI) dendritic cells show the presence of internalized Si-PEI-CpG-MPL BR5-Akt tumor cells after a one-hour incubation at 37° C. (FIG. 3A). The actin fluorescence is shown with variable thresholding to enable identification of intracellular and surface bound silicified tumor cells by dendritic cells (FIG. 3A). To specifically track silicified tumor cells (blue: actin; green: tubulin; violet: DAPI), they were loaded with fluorescent nanoparticles (red; rhodamine B labeled to function as a probe) prior to silicification (FIG. 3B). Internalization of Si-PEI-CpG-MPL, Si-PEI, or Si-only tumor cells by dendritic cells was compared to that of live or paraformaldehyde-fixed tumor cells using flow cytometry and CELL TRACE (Thermo Fisher Scientific, Inc., Waltham, MA) far red-labeled cells. Silicified tumor cells bound with CpG and MPL had a nine-fold increase in uptake compared to live or fixed tumor cells (FIG. 4A). The increase in uptake of modified tumor cells is based on the ability of TLR ligands to specifically promote phagocytosis in both murine and human cells through induction of a phagocytic gene program.

Further, tumor antigen presentation in the context of MHC I was tested using the ID8ova cell line that expresses the model antigen ovalbumin. Ova peptide (SIINFEKL, SEQ ID NO:1) presentation on MHC I by dendritic cells was assessed by flow cytometry analysis after 72 hours of co-culture with Si ID8ova tumor cells. These experiments demonstrated that cell silicification and surface modification with TLR ligands preserves the presented antigens compared to irradiation (FIG. 4B). Thus, peritoneal lymph node dendritic cells internalize dendritic cells following peritoneal administration.

While illustrated above in the context of exemplary embodiments using MPL and CpG as TLR agonists that induce uptake of silicified cells by dendritic cells, the compositions and methods described herein can involve modifying the surface of silicified cells with any TLR agonist that induces uptake by dendritic cells.

Silicified, surface modified tumor cells generate tumor-specific T cell immunity in vivo. The BR5-Akt cell line is a model cell line developed on an FVB background, facilitating in vivo imaging with the IVIS Spectrum. To evaluate the immunogenicity of surface-modified silicified tumor cells in vivo, mice were injected with silicified, surface modified BR5-Akt tumor cells prior to BR5-Akt-Luc2 tumor challenge and evaluated for evidence of a T cell response. In these experiments, female FVB mice (n=4/group, with replicate studies) received 3×10⁶ silicified tumor cells intraperitoneally three weeks prior to tumor challenge. To confirm that this effect was antigen-specific, modified mesoporous silica nanoparticles (MSNs) were used as an additional control. Modified MSNs have similar surface presentation of PEI, CpG, and MPL as silicified tumor cells but lack the tumor cell component with associated antigens. Tumor burden was monitored based on bioluminescence with IVIS Spectrum imaging and quantified as photons/second (FIG. 5). Within three to six days of tumor challenge, no bioluminescence was detectable in the silicified tumor cell group (vaccine). In contrast, the PBS-treated and MSN-treated mice showed progressive tumor growth requiring euthanasia by Day 40. These experiments demonstrated that silicified tumor cells generate the protective effects of vaccination, and that vaccination with modified MSN had no survival benefit compared with untreated controls.

As used herein, the term “protect,” “protective,” and variations thereof refer to any degree of a measurable reduction in the likelihood, frequency, extent and/or severity of the noted phenomenon—e.g., tumor burden. Therefore, the term “protect” or “protective effect” should be construed as reducing, e.g., tumor burden compared to an untreated control to any degree, and should not be construed as requiring absolute prevention of any evidence of the noted phenomenon in every instance of use.

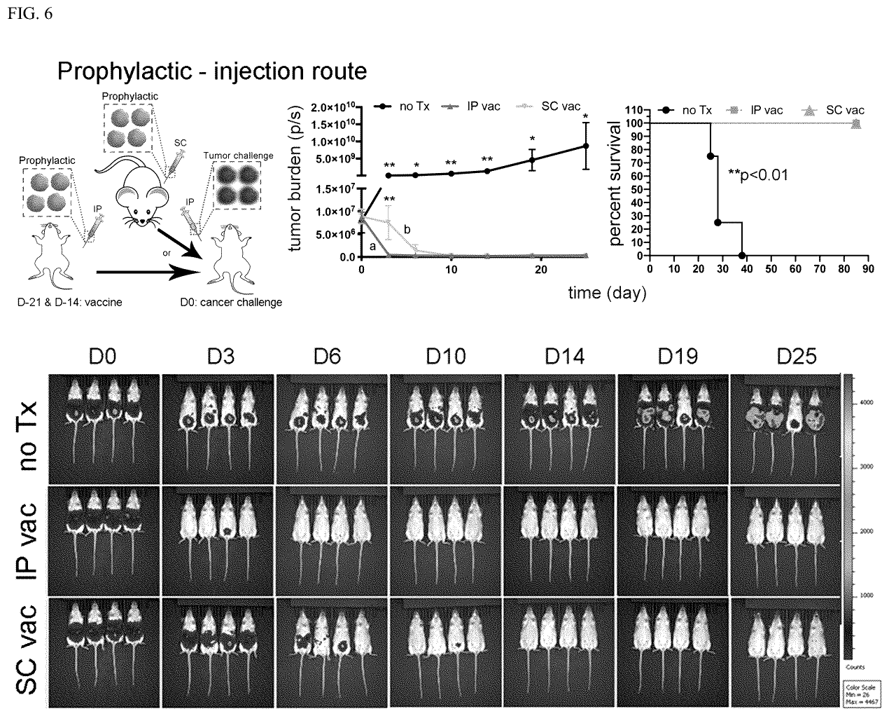

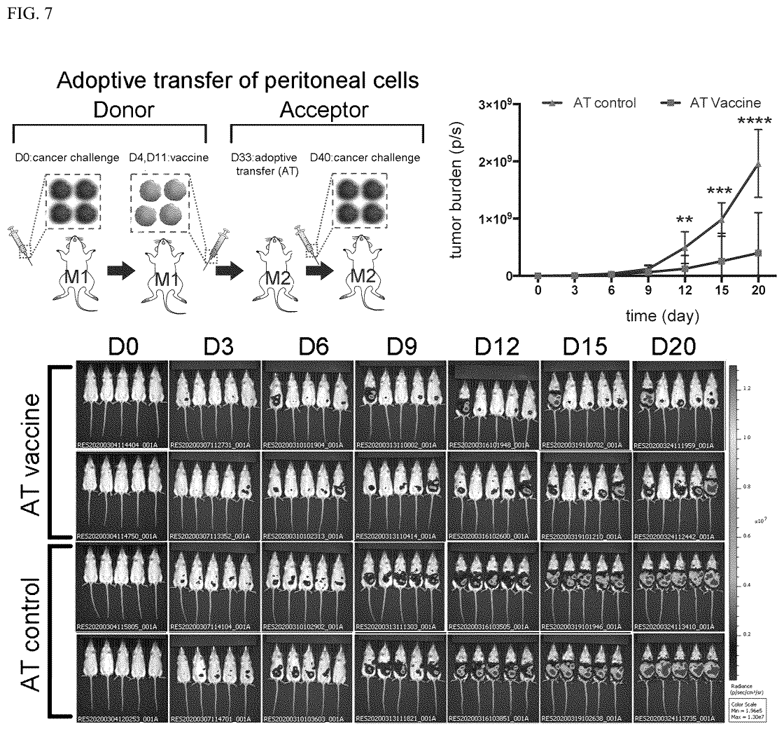

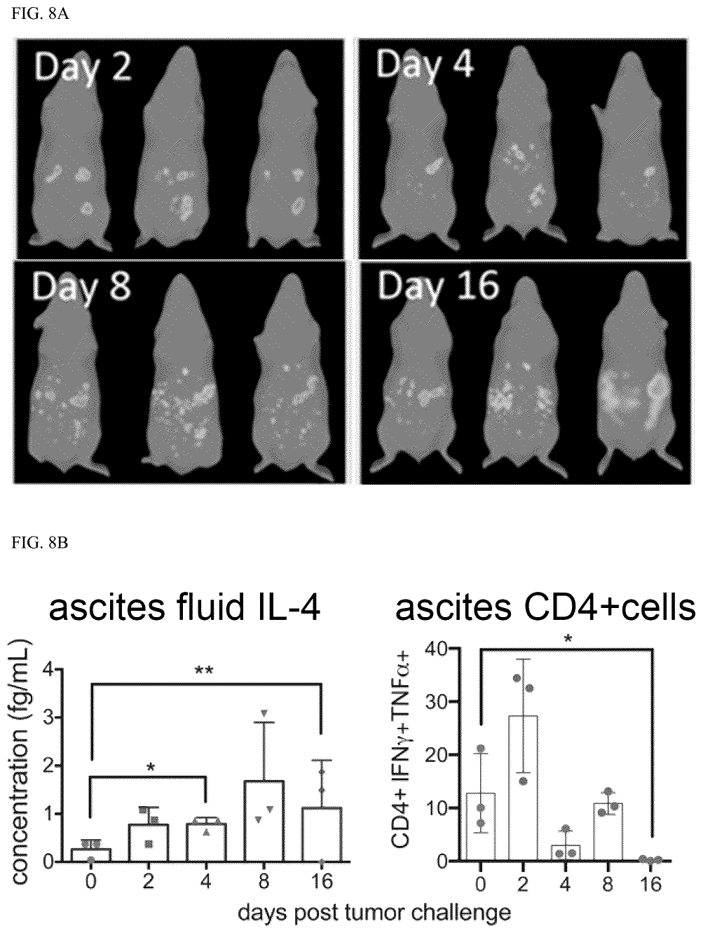

In support of silicified tumor cells generating prophylactic systemic immunological memory, naïve FVB mice were administered either two subcutaneous (SC) or two intraperitoneal (IP) doses of modified silicified tumor cells at three weeks and two weeks prior to intraperitoneal tumor challenge. While a slight delay in tumor clearance was observed following SC compared to intraperitoneal administration, all detectable tumor cells were eliminated in treated mice, resulting in durable survival of all mice beyond 85 days (FIG. 6). To test for immune memory after vaccination, 2×10⁵ magnetically enriched peritoneal CD8⁺ cells were collected from vaccinated mice on Day 33 post tumor challenge and transferred to naïve mice 24 hours prior to tumor challenge. CD8⁺ cells from naïve (unvaccinated, non-tumor experienced) mice were used as a negative control. The adoptive transfer of peritoneal CD8⁺ T cells from vaccinated mice protected recipient mice from tumor challenge—i.e., the adoptive transfer of CD8⁺ T cells from vaccinated mice reduced the tumor burden in the recipient mice compared to the tumor burden in recipient mice receiving CD8⁺ T cells from naïve mice (FIG. 7). Collectively, these results demonstrate the induction of protective T cell memory after vaccination and tumor clearance.

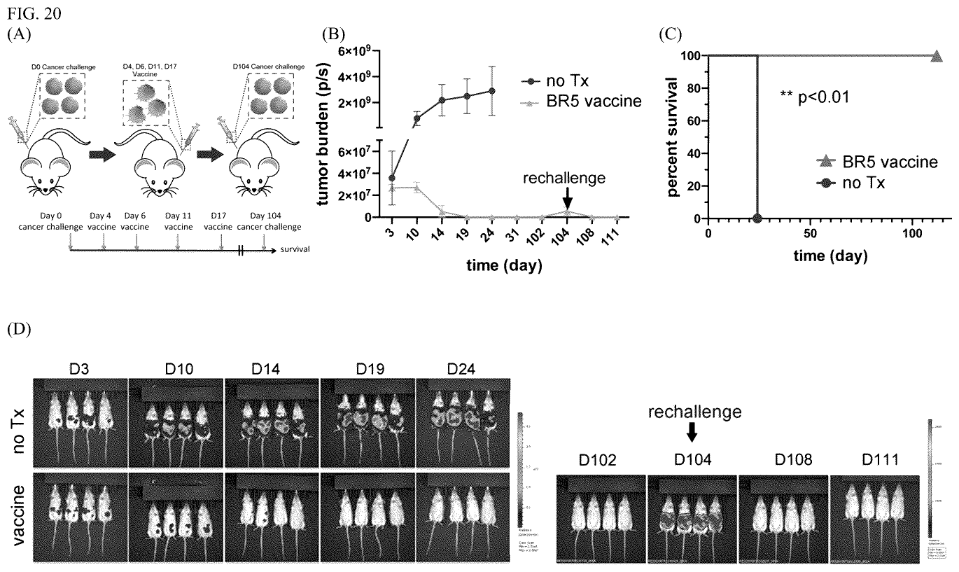

Therapeutic vaccination in vivo results in durable survival benefit. The ability of vaccination to clear established tumors was evaluated in FVB mice with BR5-Akt-Luc2 tumors (n=4/group with replicates; FIG. 20). In these experiments, mice were injected intraperitoneally with Si-tumor cells on Days 4, 6, 11, and 17 after intraperitoneal tumor challenge. Mice vaccinated with modified silicified tumor cells eliminated all detectable tumor, as evaluated by bioluminescence, resulting in durable survival beyond 100 days. To test whether therapeutic vaccination could generate a protective memory response, these mice were subjected to a second tumor challenge on Day 104 and demonstrated that they were again able to clear tumor. In summary, vaccination with silicified tumor cells has a marked therapeutic effect in this high grade serous ovarian cancer model and induces protective immunologic memory.

Different dosing and vaccination schedules were tested to optimize the therapeutic efficacy of silicified tumor cells. All dosing schedules, which included one to four doses of 3×10⁶ silicified tumor cells were effective in clearing established tumors. When silicified tumor cell number per injection was evaluated in mice receiving two doses, vaccination with 3×10⁶ or 3×10⁷ silicified tumor cells cleared tumors in all mice.

To evaluate whether intraperitoneal vaccination supports systemic immunity, mice with subcutaneous tumors, or with both subcutaneous and intraperitoneal tumors, were established. Following two intraperitoneal injections with silicified tumor cells, mice were able to clear subcutaneous tumors when tumor cells were also present in the peritoneal cavity. This indicates that vaccination in the tumor environment leads to systemic changes and peripheral anti-tumor immunity. In support of a T cell mediated response, peritoneal CD8⁺ T cells isolated from vaccinated mice were effective at ex vivo cancer cell killing in contrast to CD8⁺ T cells from naïve mice.

BR5-Akt-luciferase ovarian tumor growth over time is shown in 3D bioluminescent images following intraperitoneal injection in 6-week-old female mice. The Th2 cytokine IL-4 increases within the peritoneal fluid (ascites) with time. Conversely, expression of the immune activation markers IFNγ and TNFα in peritoneal CD4⁺ T cells decreased significantly, with almost no expression by Day 16. This data indicate that the tumor microenvironment becomes increasingly immune suppressive with time.

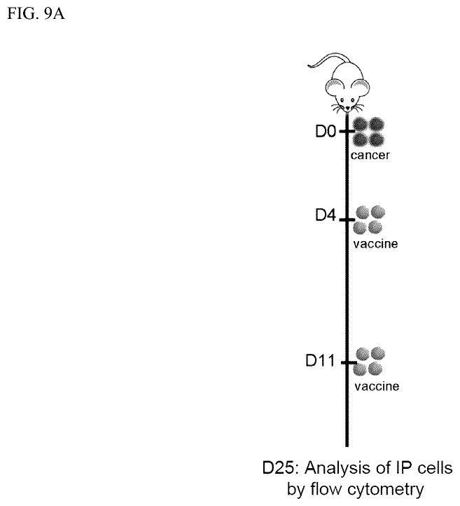

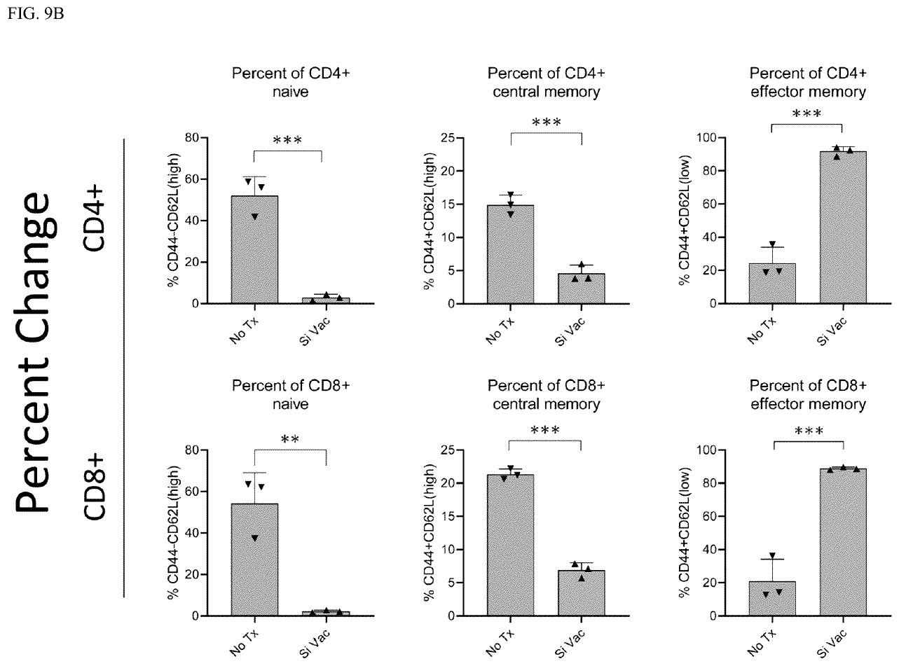

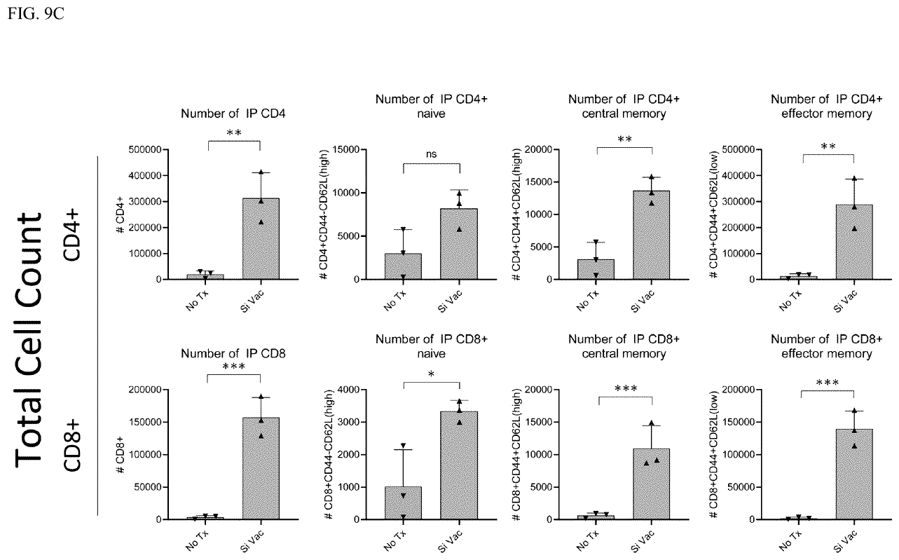

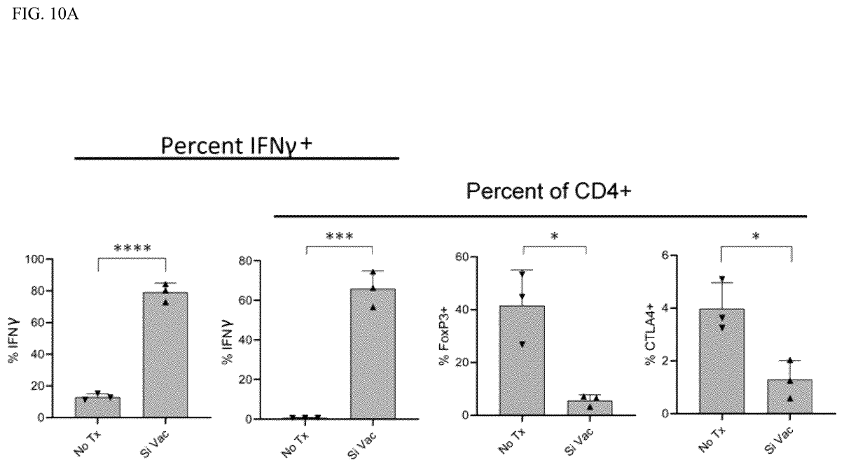

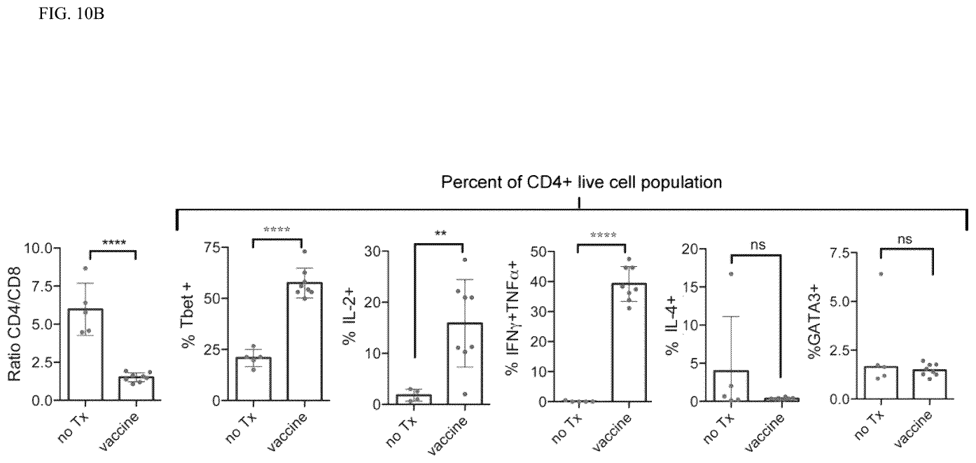

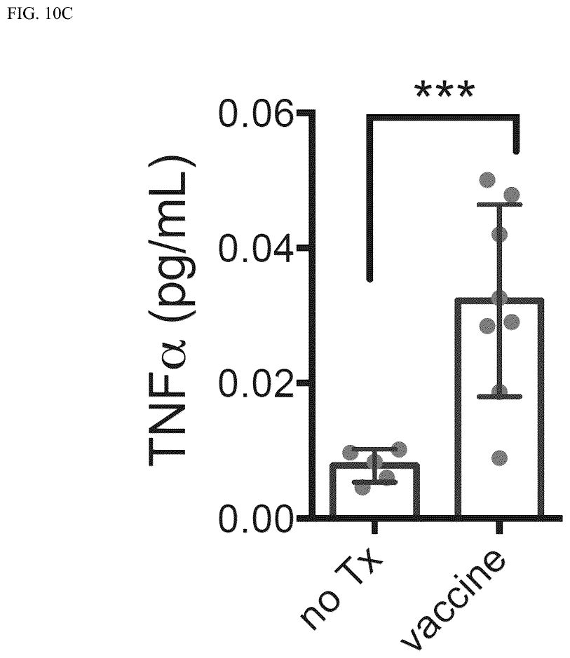

Vaccination with silicified tumor cells modulates the functional status of tumor-associated lymphocytes. Vaccinated mice had a significant increase in the total number of CD4⁺ and CD8⁺ T cells and a marked increase in the proportion of effector memory cells within these populations in the peritoneal tumor environment (FIG. 9B, 9C). In addition, while the proportion of functionally activated T cells expressing IFNγ upon ex vivo stimulation (FIG. 10A), the percentage of Tregs and CD4⁺ cells expressing the exhaustion marker CTLA-4 were lower in vaccinated mice (FIG. 10A). The most beneficial changes in the tumor microenvironment were seen with two doses of 3×10⁶ Si-tumor cells, analysis of tumor-associated lymphocytes.

To evaluate the effect of silicified tumor cell vaccination on clinically relevant endpoints, T cell phenotype and distribution were characterized in vaccinated mice. In women with ovarian cancer, a CD4/CD8 ratio of less than 1.6 correlates with improved outcomes. While silicified tumor cell vaccination increased the proportions of both CD4⁺ and CD8⁺ T cells in the peritoneal tumor environment, it reduced the mean CD4/CD8 ratio from 6.0 to 1.5 (FIG. 10B, ranges 4.5-8.7 and 1.1-1.9 for no Tx and vaccinated groups). Vaccination also significantly increased T-bet expression by intraperitoneal CD4 cells, a transcription factor associated with Th1 differentiation (FIG. 10B). These results are supported by an increase in IL-2, IFNγ, and TNFα expression in CD4⁺ cells, as well as in increase in co-expression of IFNγ, and TNFα in CD8⁺ cells, in vaccinated mice following ex vivo stimulation (FIG. 10B). Vaccination also increased TNFα levels in ascites (FIG. 10C).

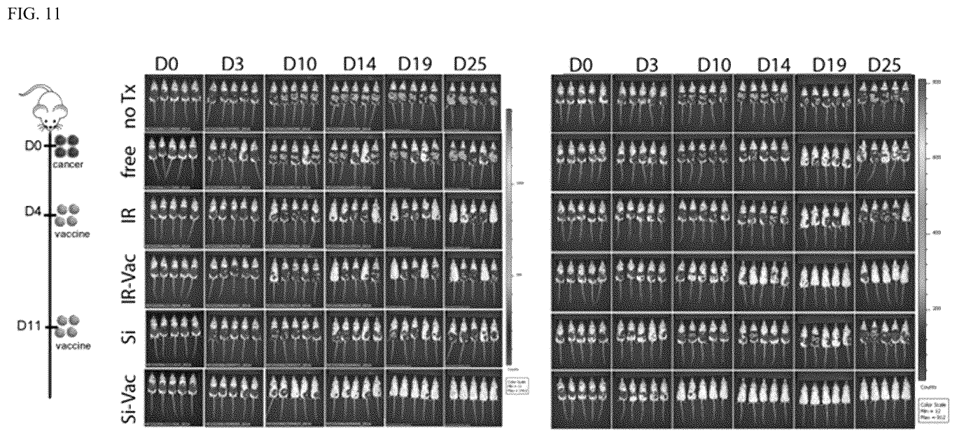

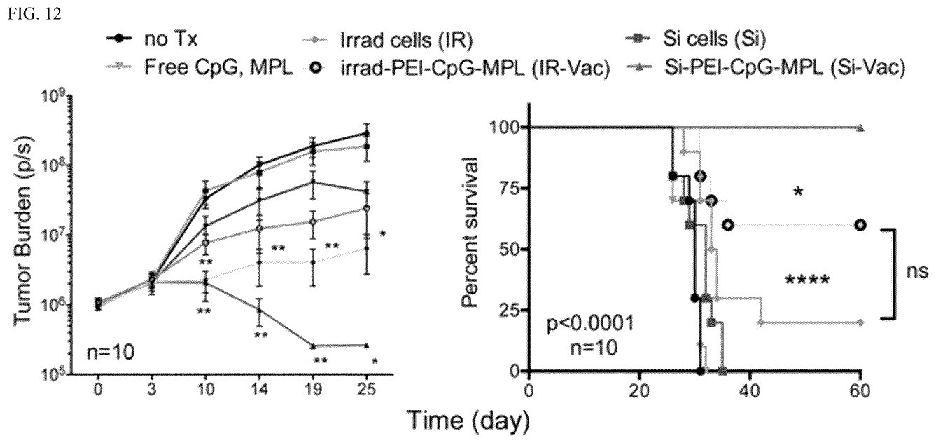

The silicified tumor cell vaccine was compared with prior strategies for cellular cancer vaccines using a previously described clinically tested protocol to vaccinate mice with irradiated cancer cells (Koster et al., 2019, Cancer Immunol Immunother 68:1025-1035). These experiments demonstrated the superiority of silicified tumor cell vaccines compared to an irradiated cancer cell vaccine (FIG. 11, FIG. 12). Inclusion of PEI, CpG, and MPL at concentrations equivalent to that on silicified tumor cells were tested as an additional control and found to be ineffective. Furthermore, silicification enables cancer cells to be dehydrated and stored at room temperature (FIG. 13A). Following rehydration, the silicified tumor cells were coated with TLR ligands or other immune agonists, enabling tailoring of immune responses through selective ligands that address the clinical situation. To demonstrate the ability of rehydrated cells to work as an effective vaccine, dehydrated, cryo-silicified cancer cells that had been stored at room temperature for two weeks were rehydrated in PBS and surface modified with PEI, CpG, and MPL. Treatment of mice having established tumors with the rehydrated vaccine eliminated all tumor cells (FIG. 13B, top, n=8 mice). Fresh and dehydrated/rehydrated silicified tumor cells were equally effective at eliminating established tumors in mice (FIG. 13B, bottom graph). In contrast, irradiated cell vaccines were unable to be rehydrated, remaining aggregated (FIG. 13A). The ability to store cellular tumor vaccines at room temperature is a significant advance that is expected to facilitate production and expand access to personalized cancer immune therapy.

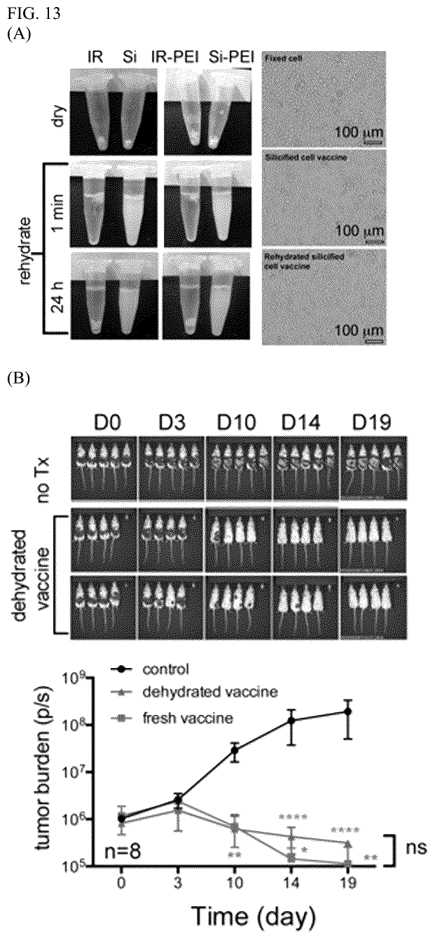

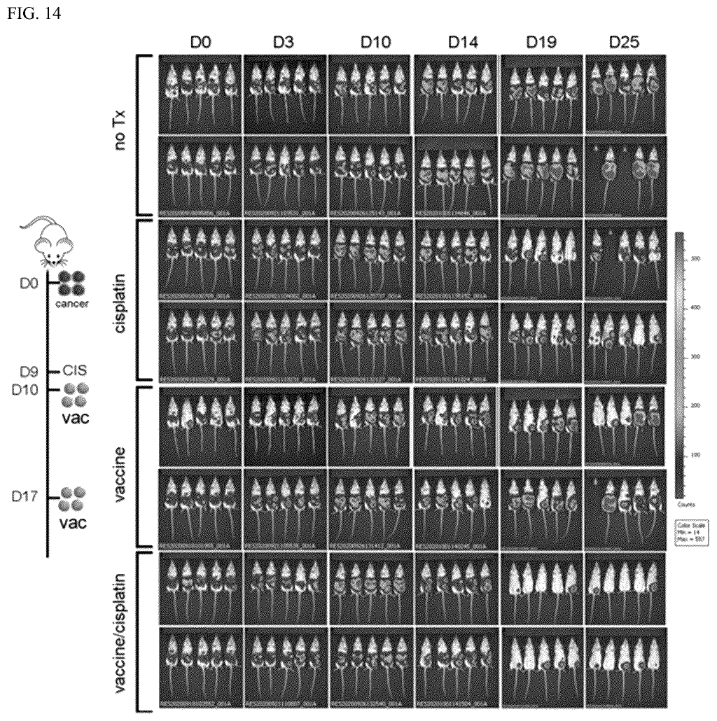

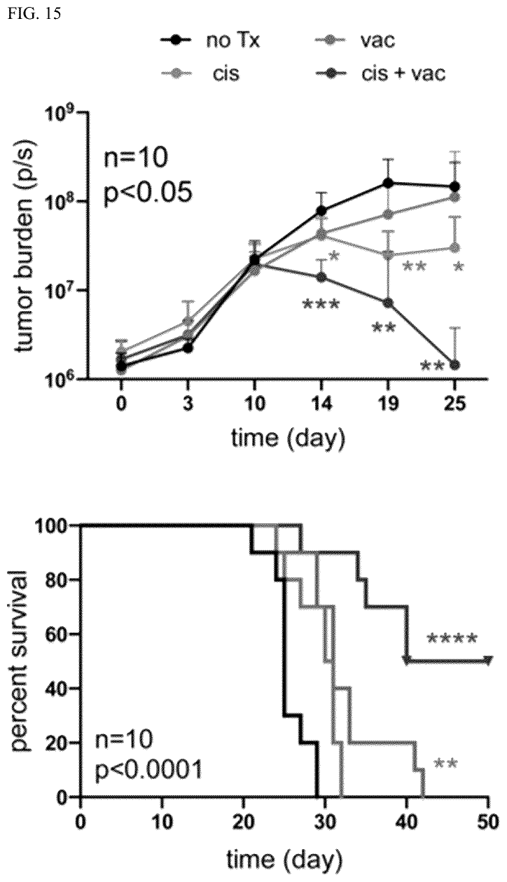

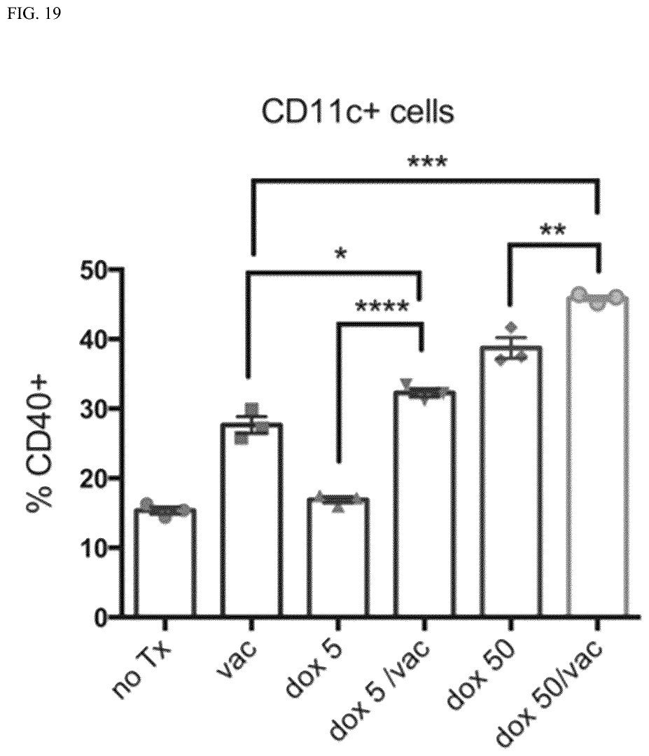

Si tumor cell vaccination enhances cisplatin efficacy in late stage ovarian cancer. Standard frontline treatment for women with ovarian cancer includes platinum-based chemotherapy. To test whether silicified tumor cell vaccination could enhance treatment outcomes following chemotherapy, tumor burden and survival were assessed in mice treated with cisplatin with or without subsequent silicified tumor cell vaccination. To account for the fact that most women with ovarian cancer present with advanced disease, treatment with cisplatin was delayed until Day 9 after tumor challenge. 2 mg/kg cisplatin was administered intraperitoneally. In one cohort, cisplatin treatment was followed by silicified tumor cell vaccination on Day 10. Tumor bioluminescence and survival were measured. Cisplatin monotherapy reduced tumor burden in some mice and improved survival (FIG. 14, FIG. 15). Vaccine monotherapy also reduced tumor burden in some mice and improved survival (FIG. 14, FIG. 15). Tumor burden was reduced in all mice receiving vaccination in addition to cisplatin, with improved overall survival (FIG. 14, FIG. 15; p<0.0001). The effects of the combination therapy were greater than the sum of effects from the two monotherapies, indicating a synergistic effect. Moreover, in vitro treatment of ovarian cancer cells with the chemotherapeutic agent doxorubicin resulted in cell culture supernatant that contained immunogenic molecules that enhanced vaccine-mediated activation of dendritic cells (FIG. 19).

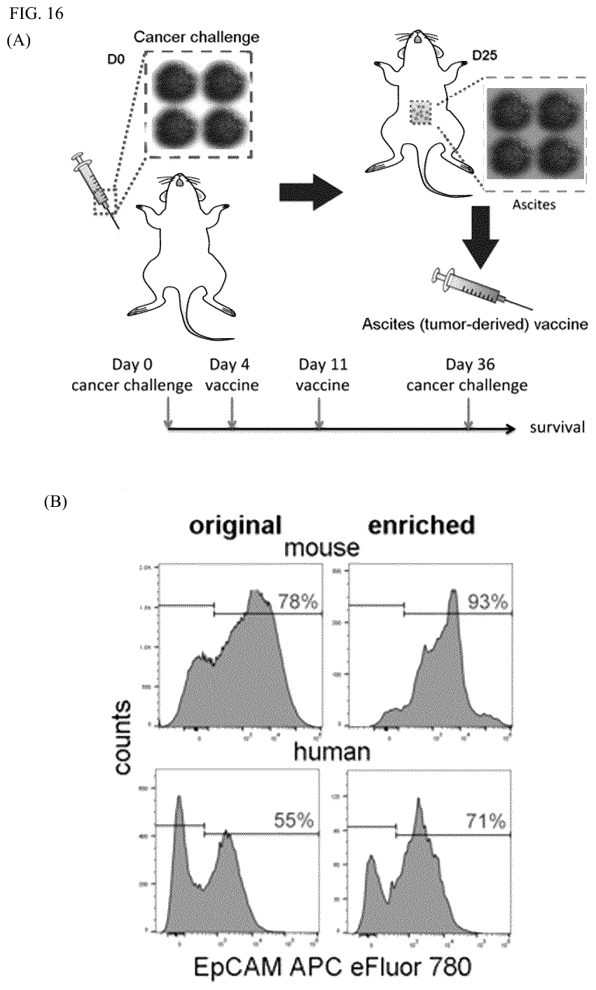

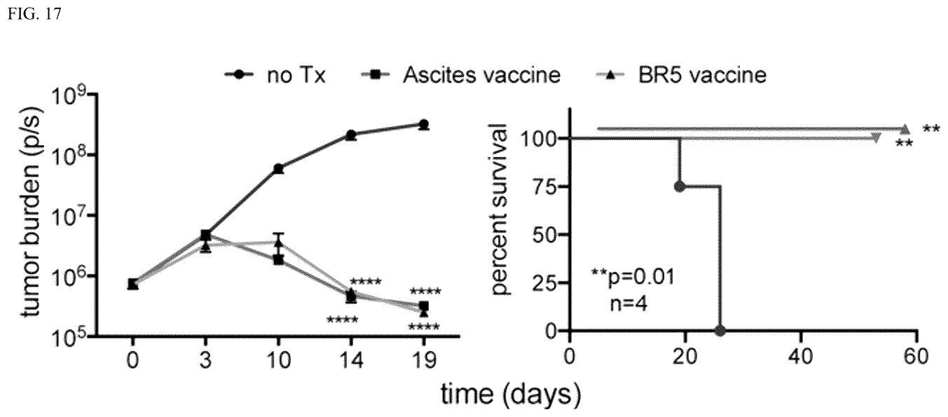

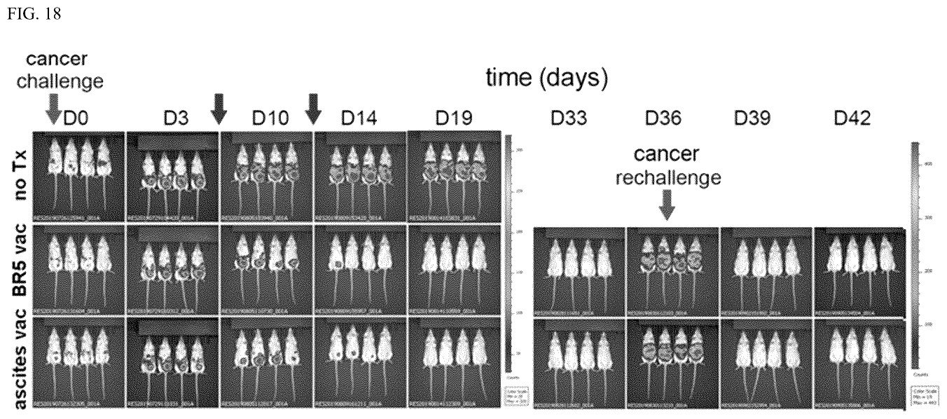

Ascites can be used for silicified tumor cell vaccine development. Creating silicified tumor cell vaccines for cancer patients requires a source of autologous tumor cells. Clinically, ovarian cancer presents at late stages of disease when patients have metastases throughout the peritoneal cavity and accumulation of malignant ascites. To evaluate translational applications of the silicified tumor cell vaccine platform, ascites cells were collected from mice with late stage BR5-Akt tumors for silicification (FIG. 16A). Tumor cells from mouse or human ascites were enriched by filtration capture and silicified using the protocol developed using cancer cell lines. Ascites displayed increased proportions of EpCAM⁺ cancer cells following filtration (FIG. 16B). Vaccination using silicified mouse ascites tumor cells at the same dose and schedule optimized previously demonstrated equivalent efficacy as vaccination using silicified tumor cells grown in vitro (FIG. 17, FIG. 18). Secondary tumor challenge on Day 36 was rapidly cleared. These results indicate that vaccine production is feasible using available tumor samples to create personalized vaccines.

Vaccination with silicified tumor cells is not associated with significant immune-related toxicity. Immune toxicity in response to vaccination was assessed using blood and tumor samples collected on Day 19. No significant differences in complete blood count or electrolyte levels were observed in vaccine recipients compared with untreated controls. Consistent with a T cell mediated immune response, treatment of tumor-bearing mice with 1-2 doses of silicified tumor cells significantly increased lymphocytes. Importantly, vaccination had no impact on renal or hepatic function. Finally, none of the vaccinated mice developed rash, alopecia, diarrhea, or weight loss suggestive of treatment toxicity.

Histopathologic analysis performed by a board certified veterinary pathologist showed pronounced necrosis in small residual islands of tumor in vaccinated mice associated with marked immune cell infiltration. Similarly, higher numbers of leukocytes were noted in peritoneal samples from vaccinated mice but no evidence of suppurative peritonitis was found.

This disclosure therefore describes a modular vaccine platform for personalized immune therapy that demonstrates durable therapeutic efficacy in high grade serous ovarian cancer models. A personalized silicified cell vaccine can be produced within 24 hours, substantially reducing the time required for cell culture and ex vivo modification for existing cell-based tumor vaccines. Once silicified, cells are stable, enabling long term dry storage without cryopreservation. The dried silicified cell vaccine can then be reconstituted prior to being administered to a subject. These features of the silicified cell vaccines described herein address a global need for versatile tumor vaccines with the potential to reduce existing disparities in access to cancer immune therapy. As a result, this platform integration of immune therapy into cancer treatment protocols.

Moreover, the silicified tumor cell vaccine can act synergistically with other cancer therapies including, but not limited to, chemotherapies. For example, this disclosure demonstrates that silicified tumor cell vaccination enhances survival in combination with cisplatin, which is considered the backbone of frontline treatment for ovarian cancer. These results demonstrate that Si cell vaccination can be effectively integrated into the established standard of care management for various cancers and reduce the likelihood and/or severity of recurrence, thereby enhancing patient outcomes.

As used herein, “silicified cell” and variants thereof (e.g., “silicified tumor cell” etc.) refer collectively to a silicified cell or a silicified cell fragment or silicified cell-derived body, such as, for example, a silicified exosome, a silicified microvesicle, or a silicified apoptotic body. Exemplary tumor cells include cells derived from patient tumors (autologous or allogenic), blood, ascites, established tumor cell lines, or de-differentiated cells.

The silicified cell may be formulated with a pharmaceutically acceptable carrier to form a pharmaceutical composition. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with a silicified cell without causing any undesirable biological effects or interacting in a deleterious manner with any of the other components of the pharmaceutical composition in which it is contained.

A silicified cell may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical can be administered via a sustained or delayed release.

Thus, a silicified cell may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, solution and the like. The formulation may further include one or more additives including such as, for example, an adjuvant. Exemplary adjuvants include, for example, pathogen-associated molecular patterns (PAMPs), such as Toll-like receptor (TLR) ligands, damage-associated molecular patterns (DAMPs), cytokines, proteins, carbohydrates, lectins, Freund's adjuvant, aluminum hydroxide, or aluminum phosphate.

A formulation may be conveniently presented in unit dosage form and may be prepared by methods well known in the art of pharmacy. Methods of preparing a composition with a pharmaceutically acceptable carrier include the step of bringing the silicified cell into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of silicified cell administered can vary depending on various factors including, but not limited to, the specific silicified cell being administered, the weight, physical condition, and/or age of the subject, and/or the route of administration. Thus, the absolute amount of silicified cell included in a given unit dosage form can vary widely, and depends upon factors such as the species, age, weight and physical condition of the subject, and/or the method of administration. Accordingly, it is not practical to set forth generally the amount that constitutes an amount of silicified cell effective for all possible applications. Those of ordinary skill in the art, however, can readily determine the appropriate amount with due consideration of such factors.

In some embodiments, the method can include administering sufficient silicified cells to provide a dose of, for example, from about 50 silicified cells/kg to about 1×10¹⁰ silicified cells/kg to the subject, although in some embodiments the methods may be performed by administering the silicified cells in a dose outside this range. In some of these embodiments, the method includes administering sufficient silicified cells to provide a dose of from about 100 silicified cells/kg to 1×10⁹ silicified cells/kg to the subject, for example, a dose of from about 1000 silicified cells/kg to about 10,000 silicified cells/kg.

A single dose may be administered all at once, continuously for a prescribed period of time, or in multiple discrete administrations. When multiple administrations are used, the amount of each administration may be the same or different. For example, a dose of 10⁶ silicified cells in a day may be administered as a single administration of 10⁶ silicified cells, continuously over 24 hours, as two administrations of 5×10⁵ silicified cells, or as a first administration of 7.5×10⁵ silicified cells followed by a second administration of 2.5×10⁵ silicified cells. When multiple administrations are used to deliver a single dose, the interval between administrations may be the same or different.

In some embodiments, the active agent may be administered, for example, from a single dose to multiple doses per week, although in some embodiments the method can involve a course of treatment that includes administering doses of the active agent at a frequency outside this range. When a course of treatment involves administering multiple doses within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

The silicified cells described herein can be used to treat a subject having, or at risk of having, a condition for which treatment is intended. That is, the treatment may be therapeutic or prophylactic. Treatment that is prophylactic—e.g., initiated before a subject manifests a symptom or clinical sign of the condition for which treatment is intended such as, for example, while neoplasia remains subclinical or while a subject may be in remission—is referred to herein as treatment of a subject that is “at risk” of having the condition. As used herein, the term “at risk” refers to a subject that may or may not actually possess the described risk. Thus, for example, a subject “at risk” of developing a tumor is a subject possessing one or more risk factors associated with developing the tumor such as, for example, genetic predisposition, ancestry, age, sex, geographical location, lifestyle, or medical history, including prior treatment for a tumor.

Accordingly, a composition can be administered before, during, or after the subject first exhibits a symptom or clinical sign of the condition for which treatment is intended. Treatment initiated before the subject first exhibits a symptom or clinical sign of the condition may result in decreasing the likelihood that the subject experiences clinical evidence of the condition compared to a similarly situated subject to whom the composition is not administered, decreasing the severity of symptoms and/or clinical signs of the condition, and/or completely resolving the condition. Treatment initiated after the subject first exhibits a symptom or clinical sign of the condition for which treatment is intended may result in decreasing the severity of symptoms and/or clinical signs of the condition compared to a similarly situated subject to whom the composition is not administered, and/or completely resolving the condition.

Thus, the method includes administering an effective amount of the composition to a subject having, or at risk of having, a condition for which treatment is intended. In this aspect, an “effective amount” is an amount effective to reduce, limit progression, ameliorate, or resolve, to any extent, a symptom or clinical sign related to the condition.

In certain embodiments, the method can further include surgically resecting the tumor and/or reducing the size of the tumor through chemical (e.g., chemotherapeutic) and/or radiation therapy. Exemplary tumors that may be treated include tumors associated with ovarian cancer, cervical cancer, lung cancer, colon cancer, rectum cancer, urinary bladder cancer, melanoma, kidney cancer, renal cancer, oral cavity cancer, pharynx cancer, pancreas cancer, uterine cancer, thyroid cancer, skin cancer, head and neck cancer, prostate cancer, and/or hematopoietic cancer.

As used herein, a “subject” can be any animal such as, for example, a mammal (e.g., dog, cat, horse, cow, sheep, goat, monkey, etc.). In certain embodiments, the subject can be a human.

In some embodiments, the method further includes administering one or more additional tumor therapies. The tumor therapy can involve, for example, radiation therapy, one ore more therapeutic agents, or one or more imaging agents administered to monitor, for example, biodistribution of a therapeutic agent or a therapeutic response. An additional tumor therapy can be administered before, after, and/or coincident to the administration of a silicified cell vaccine. A silicified cell vaccine and the additional tumor therapy (e.g., therapeutic agents) may be co-administered. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. When the co-administered components are capable of being administered in a composition, simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture or physical mixture with one another. In other embodiments, the silicified cell vaccine and the additional tumor therapy, when capable of be administered as a composition, may be administered as part of a mixture or cocktail. In some aspects, the administration of silicified cell vaccine may allow for the effectiveness of a lower dosage of other therapeutic modalities when compared to the administration of the other therapeutic agent or agents alone, thereby decreasing the likelihood, severity, and/or extent of the toxicity observed when a higher dose of the other therapeutic agent or agents is administered.

Exemplary additional therapeutic agents include altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamaide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, and vinorelbine.

In some embodiments, of the method can include administering sufficient silicified cell vaccine as described herein and administering the at least one additional therapeutic agent provides therapeutic synergy. In some aspects of the methods of the present invention, a measurement of response to treatment observed after administering both a silicified cell vaccine as described herein and the additional therapeutic agent is improved over the same measurement of response to treatment observed after administering either the silicified cell vaccine or the additional therapeutic agent alone.

In the preceding description and following claims, the term “and/or” means one or all of the listed elements or a combination of any two or more of the listed elements; the terms “comprises,” “comprising,” and variations thereof are to be construed as open ended i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Materials

### Laboratory Grade Materials

Low molecular weight chitosan, poly-L-lysine, puromycin dihydrochloride, rhodamine B isothiocyanate mixed isomers, and 10% buffered formalin, were purchased from Sigma-Aldrich (St. Louis, MO). PROLONG gold antifade mountant with DAPI, ALEXA FLUOR 488, alpha tubulin antibody, phosphate-buffered saline (PBS), and RPMI 1640 were purchased from Thermo Fisher Scientific (Waltham, MA). Fetal bovine serum (FBS) was purchased from American Tissue Culture Collection (ATCC, Manassas, VA). 0.05% EDTA trypsin solution, penicillin-streptomycin, and rhodamine or ALEXA FLUOR 647 phalloidin were purchased from Life Technologies Corporation (Carlsbad, CA). Dulbecco's Modified Eagle's Medium (DMEM) was obtained from Caisson Labs (Smithfield, UT). CELL TITER-GLO 2.0 Assay was purchased from Promega (Madison, WI). Recombinant murine granulocyte macrophage colony stimulating factor (GM-CSF) was purchased from R&D Systems (Minneapolis, MN). XENOLIGHT D-luciferin potassium salt was purchased from Perkin Elmer (Boston, MA). Reversible Strainers (37 μm mesh) were purchased from Stemcell Technologies, Inc. (Vancouver, Canada).

### Antibodies

CD11c FITC (HL3), CD326 (EpCAM, G8.8) APC, CD326 (MaH EpCAM IB7) eFluor 660, CD3 (17A2) APC-eFluor 780, CD4 (GK1.5) APC, CD8a (53-6.7) eFluor 450 and Alexa Flour 488, CD11b (M1/70) APC and FITC, CD11c (N418) PerCP-Cyanine5.5 and PECy7, CD40 (3/23) PE, CD44 (IM7) PerCP-Cyanine5.5, CD62L (L-selectin, MEL 14) FITC, CD152 (CTLA-4, UC10-4B9) PE and PerCp-Cy5.5, CD223 (LAG3, C9B7W) PerCP-Cy5.5 BD, CD279 (PD-1, J43) PE-Cyanine7, FOXP3 (FJK-16s) PE, IFNγ (XMG1.2) Alexa Fluor 488, MHC Class II (I A/I E) (M5/114.15.2) FITC, RORγT PE (AFKJS-9), GATA-3 PerCP-eFlour 710 (TWAJ), TNFα PerCP-eFlour 710 (MP6-XT22), CD45R (B220) FITC, Fc receptor blockers (anti-CD16/CD32 (clone 2.4G2)), mouse IgG (31205), and LIVE/DEAD Fixable Aqua Dead Cell Stain Kit for 405 nm excitation were purchased from eBioscience, Inc. (San Diego, CA) and Thermo Fisher Scientific, Inc. (Waltham, MA).

IL-4—APC (11B11), CD152 (CTLA-4) PerCP, TIM3 (B8.2C12) APC were purchased from BioLegend (San Diego, CA).

IL-2 PE (JES6-5H4) was purchased from BD Biosciences (San Jose, CA).

### Methods

### Cell Lines and Mouse Models of Ovarian Cancer

The BRCA1-deficient BR5-Akt cell line, generated on an FVB background, was previously described (Xing, D. & Orsula, S., 2006, Cancer Res 66:8949-8953). The ID8ova cell line, generated from C57BL/6 ovarian epithelial cells, and transfected to express ovalbumin constitutively, was previously described (Roby, K. F., 2000, Carcinogenesis 21:585-591). Both ID8ova and BR5-Akt cell lines are syngeneic models of high-grade serous epithelial ovarian cancer. To monitor tumor burden using a bioluminescent tag, the cell lines were lentivirus transduced to constitutively express firefly2 luciferase. Cell lines were cultured in DMEM containing 10% FBS and 100 units/100 μg penicillin/streptomycin at 37° C. and 5% CO2. Trypsin-EDTA was used to harvest cells.

To prepare bone marrow-derived dendritic cells (DC), bone marrow was harvested from the femurs of female murine C57BL/6 or FVB mice using a 27 gauge needle and syringe to flush the marrow from the bone. RBC were lysed with BD Lysis buffer as described by the vendor. Cells were cultured in six-well plates (3 ml/well) for eight to ten days in RPMI 1640 medium supplemented with 10% FBS, 100 mM β-mercaptoethanol, penicillin/streptomycin, and 10 ng/mL recombinant murine GM-CSF. Half of the media was replaced every two to three days with fresh media and cytokines.

Mice were purchased from Charles River Laboratories (Wilmington, MA) and housed in a specific pathogen-free facility. All animal protocols were approved by the Institutional Animal Care and Use Committee (IACUC) at the University of New Mexico (Albuquerque, NM). To generate consistent engraftment and predictable disease progression, 2×10⁵ BR5-Akt-Luc2 cells in 200 μL PBS were administered by intraperitoneal (IP) injection in 6-7-week-old FVB female mice as previously described (Higuchi et al., 2015, Cancer Immunol Res 3:1257-1268). Mice were sacrificed when moribund or when weight reached 30 g due to ascites accumulation. Mice were monitored and weighed every two to three days. For studies that included subcutaneous (SC) tumors, female mice were injected with 200 μl PBS containing 2×10⁵ BR5-Akt-Luc2 cells on the dorsal surface using isoflurane as an inhalation anesthetic.

### Cell Silicification

3×10⁶ BR5-Akt or ID8ova cells were washed with PBS, followed by physiological saline (154 mM NaCl), and then suspended in 1 mL silicic acid solution containing 10 mM TMOS, 100 mM NaCl and 1.0 mM HCl (pH 3.0), with scale up as needed. Optimization of conditions for biological use evaluated cell stability and dispersion following silicification in 5 mM-100 mM TMOS, and 100 mM verses 154 mM NaCl. Following a 5-10-minute incubation at room temperature, the cell suspension was transferred to −80° C. for 24 hours. Silicified cells were then washed with endotoxin-free water, followed by PBS. To compare Si content with published cell silicification techniques, cells were also silicified at room temperature in silicic acid solution containing 100 mM TMOS, 154 mM NaCl and 1.0 mM HCl (pH 3.0) for 24 hours as previously described (Kaehr et al., 2012, Proc Natl Acad Sci USA 109:17336-17341).

Coating Silicified Cells with Cationic Polymer

Silicified cells were made cationic using chitosan, poly-L-lysine or PEI. 3×10⁶ silicified cells were washed with water, followed by PBS, and then suspended in 1 mL of 0.2 mg/mL PEI, 2 mg/mL chitosan, or 1 mg/mL poly-L-lysine in PBS. Following 10 minutes (or as indicated) of rotation at room temperature, cells were washed twice with PBS and zeta potentials evaluated.

### Fluorescent PEI Synthesis

PEI (5 g, 0.2 mmol) was dissolved in 5 mL ethanol and Cy3-NHS (10 mg/mL in DMF, 150 μL, 2 μmol) were added. The solution was rotated at 40° C. for four days. The mixture was concentrated using a rotary evaporator, then 50 μL DMF were added to dissolve any unreacted dye. The mixture was centrifuged at 21,000 g for 20 minutes and the isolated pellet was dissolved in ethanol and transferred to the rotavapor to remove DMF traces. After one hour, the PEI-Cy3 were dissolved in PBS at 0.5 mg/mL.

### Adsorption of TLR Ligand to Silicified (Si) or Irradiated Cells

12×10⁶ silicified cells, with or without polymer coating, were washed with PBS and then suspended in 25 μL of 1 mg/mL MPL in DMSO. After a 10-minute incubation at room temperature, Si-PEI-MPL or Si-MPL cells (or their chitosan counterparts) were washed with PBS by centrifugation at 2000×g for five minutes followed by suspension in PBS. 1826 oligodeoxynucleotide adsorption followed a similar protocol using 20 μL of 2 mg/mL CpG in endotoxin-free water for every 12×10⁶ silicified cells. For dual adsorption of MPL and CpG, CpG was introduced first for 10 minutes, followed by the addition of MPL for an additional 10 minutes. To quantitate ligand loading, unbound fluorescent TLR ligand was measured using a microplate reader with excitation/emission at 470/560 nm for PEI-Cy3, and excitation/emission at 488/528 nm for CpG-FITC detection. MPL was quantified by absorption at 290 nm using a NANODROP 2000 spectrophotometer (Thermo Fisher Scientific, Inc., Waltham, MA). Irradiated BR5-Akt cells were incubated with PEI, CpG, and MPL using the same conditions optimized for Si-cells.

### Dehydration and Rehydration of Vaccine Cells

Silicified or irradiated cells (with or without PEI) were rinsed with PBS and then dried under vacuum at room temperature for 16 hours. Samples were stored at room temperature for 14 days. Prior to use, cells were rehydrated in PBS with vortexing and coated with PEI, CpG, or MPL.

### Zeta Potential Measurements

Zeta potential measurements were performed using the Malvern Zetasizer Nano-ZS (Westborough, MA) equipped with a He—Ne laser (633 nm) and non-invasive backscatter optics (NIBS). Cells were suspended in 5 mM NaCl solution with measurements performed using the monomodal analysis tool. All reported values correspond to the average of at least three independent samples.

### Optical Microscopy

For bright field imaging, cells were suspended in the water or PBS and imaged using the Nikon eclipse TS 100 inverted microscope equipped with a Nikon digital-sight DS-L3 camera.

### In Vitro Dendritic Cells Internalization of Fluorescent Silicified Cells

To image dendritic cells association with silicified cells, BR5-Akt cancer cells were first incubated with fluorescent mesoporous silica nanoparticles labeled with Cy3 or DyLight 488 immunogenic protocells (i.e., MPL presenting lipid-coated MSN) for 4-24 hours. Tumor cells were then silicified using optimized conditions and surface-masked with TLR ligands (as indicated). Dendritic cells were seeded onto glass coverslips in six-well plates at a density of 5×10⁵ cells per well and the next day, fluorescent silicified vaccine cells were added and dendritic cells were incubated as indicated. Dendritic cells were then washed with PBS and fixed with 4% paraformaldehyde for 15 minutes at room temperature followed by overnight incubation at 4° C. The following day, cells were washed with PBS, permeabilized with 0.1% Triton-X in PBS for 15 minutes, blocked with 1% BSA for 20 minutes, and then labeled with Alexa Fluor 647 phalloidin and in 1% BSA for one hour. After a final wash in PBS, coverslips were mounted on slides using Prolong Gold with DAPI. Images were acquired using a 63×/1.4NA oil objective in sequential scanning mode using a Leica TCS SP8 confocal microscope.

DC uptake of silicified cells was quantified using a flow cytometer (ATTUNE NXT, Thermo Fisher Scientific, Inc., Waltham, MA). BR5-Akt cells were stained with CELL TRACE far red (Thermo Fisher Scientific, Inc., Waltham, MA) prior to silicification. Silicified cells, surface modified with TLR ligands as indicated, were co-cultured with CELL TRACE violet (Thermo Fisher Scientific, Inc., Waltham, MA)-labeled dendritic cells for one to four hours, and then analyzed by flow cytometry for double-positive cell populations.

### T Cell Killing Assay

BR5-Akt cells were seeded into a 96-well plate at 1×10³ cells per well in 200 μl followed by overnight incubation at 37° C. in 5% CO2. Peritoneal T cells were purified from vaccinated (Day 57) or naive FVB mice using a CD8a⁺ T cell isolation kit (Miltenyi Biotec, Bergisch Gladbach, Germany). T cells were added to cancer cells at a ratio of 5:1 and the cell impermeant nuclear dye YOYO-3 iodide (Invitrogen, Carlsbad, CA; 1 mM in DMSO) was added at a dilution of 1:4000. Cells were put in the INCUCYTE live cell imaging system (Sigma-Aldrich, St. Louis, MO) and imaged for 48 hours with images acquired every two hours.

### Scanning Electron Microscopy (SEM) and Energy Dispersive X-Ray (EDX) Analysis

Silicified tumor cells were suspended in 100% ethanol and then dropped onto 5×5 mm glass slides. The glass slides were then mounted on SEM stubs using conductive adhesive tape. SEM and EDX images were acquired under high vacuum at 7.5 k using a FEI Quanta 3D Dualbeam FIB-FEGSEM with EDAX SDD EDS detector (Thermo Fisher Scientific, Inc., Waltham, MA).

### Inductively Coupled Plasma-Optical Emission Spectroscopy (ICP-OES)

ICP-OES was used to measure Si concentration in silicified cells. 20×10⁶ cells were washed with water and dried under vacuum for seven days and then mineralized in aqua regia (1:3 mixture of ultrapure HNO₃ and HCl) with a Digi prep MS SCP Science block digester at 95° C. for four hours. The digested samples were diluted and passed through 0.45 m filter. The concentration of Si was then measured using a PerkinElmer Optima 5300DV ICP-OES, with a detection limit of <0.5 mg/L. ICP-OES is calibrated with a five-point calibration curve. QA/QC measurements were also obtained to ensure quality results.

### Silicified Cell Degradation in Simulated Endosomal Solution

Twenty million silicified cells were suspended in 4 mL simulated endosomal solution containing acetate buffer, pH 5.2 (Poly Scientific R&D Corp., Bayshore, NY) and 20% FBS at room temperature under rotation for three days. After incubation, the cells were rinsed with water and then dried under vacuum for seven days. The Si content of silicified cells pre-treatment and post-treatment was measured using ICP-OES.

### Proliferation Assays

Native and silicified cells, with/without PEI coating, were assessed for cell growth using the CELLTITER-GLO 2.0 assay (Promega, Madison, WI. Briefly, cells were seeded at a density of 100,000 cells/mL in culture media in white opaque 96-well plates. After 24 hours, CELLTITER-GLO 2.0 Reagent was added to each well, and following a 10-minute incubation, luminescence was determined using a microplate reader. Percent cell viability was calculated relative to control, non-treated cells.

### Preparation of Mesoporous Silica Nanoparticles (MSNs)

A mixture of water (100 mL), ethanol (40 mL), sodium hydroxide (NaOH, 2M, 0.75 mL) and cetyltrimethylammonium bromide (CTAB, 0.640 g) was heated to 70° C. under vigorous stirring (750 rpm) in a round bottom flask immersed in an oil bath. Afterwards, tetraethyl orthosilicate (TEOS, 1 mL) was added dropwise to the solution. The TEOS was allowed to undergo a series of hydrolysis condensation reactions for two hours to yield silica CTAB-templated silica nanoparticles. The particles were then isolated by centrifugation (2000×g, 20 minutes) and then washed with methanol three times. The surfactant was removed by suspending the nanoparticles in a solution of 0.45 g/L ammonium nitrate in ethanol and stirring at 60° C. for 20 minutes. Finally, the template-free MSNs were consecutively washed twice with water and ethanol and stored suspended in ethanol. MSNs (0.5 mg) were rinsed twice with water and then suspended in 1 mL of 0.2 mg/mL PEI in PBS solution. After 10 minutes of rotation at room temperature to allow PEI binding on the MSN surface, the MSNs with PEI coating (MSN-PEI) were then rinsed with PBS twice. MSN-PEI (0.87 mg) were suspended in 20 μL of 2 mg/mL CpG in double distilled water solution. After a 10-minute incubation at room temperature, 25 μL of 1 mg/mL MPL in DMSO solution was added and incubated another 10 minutes. MSN-PEI-CpG-MPL particles were then centrifuge at 20000 relative centrifugal force (rcf) for five minutes to remove extra free ligand, and then resuspended and stored in 1 mL PBS. These conditions result in similar CpG and MPL dose content to vaccine cells.

### In Vitro Dendritic Cells Functional Studies

Dendritic cells were seeded in 12-well plates at a density of 1×10⁵ cells per well. After 24 hours, the media was removed and replaced with 2 mL of fresh complete media supplemented with 100,000 Si-PEI-CpG-MPL (or irradiated PEI-CpG-MPL) ID8ova cells for 72 hours. Alternatively, dendritic cells were incubated with Si-LPS or Si-MPL ID8ova cells for 72 hours. Irradiated cells were suspended in PBS in microfuge tubes at 3×10⁶ cells/mL and exposed to 100 Gy using an irradiation system (FAXITRON MULTIRAD, Hologic, Inc., Marlborough, MA) at 22 rV, 15 mA, 48 Gy/min. Dendritic cells were collected using 3 mM EDTA. The suspended cells were centrifuged, washed with PBS containing 1% BSA, and labeled with fluorescent antibodies specific for CD11c and either co-stimulatory molecules or SIINFEKL-H2-k0. Cells were analyzed using ta flow cytometer (FORTESSA or CALIBUR, Becton Dickinson and Co., Franklin Lakes, NJ.

Vaccination of Mice with Silicified (Si)s-Tumor Cells

Tumor-bearing or naïve female FVB mice were vaccinated intraperitoneally with irradiated (100 Gy at 225V, 15 mA, 48 Gy/min; or a Cs-137 gamma irradiator with a dose rate of 63 cGy/min) or silicified BR5-Akt (or BR5-Akt-Luc2 for vaccine viability analysis) cells (with TLR ligands as indicated) using doses of 3×10⁴, 3×10⁵, 3×10⁶, or 3×10⁷ Si-cells/mouse in 200 μl of PBS at the indicated schedules. Alternatively, mice were vaccinated subcutaneously (SC) with 3×10⁶ Si-cells by scruffing the skin at the back of their neck and injecting an equivalent volume into the loose fold of skin. Mice that cleared all tumor cells based on IVIS Spectrum bioluminescent imaging were re-challenged with 2×10⁵ BR5-Akt-Luc2 cancer cells at a later date, as indicated for each study. All control (no Tx) mice received sham PBS injections (200 μl/mouse). Single agent or combination therapy with cisplatin used IP administration of cis-diamineplatinum (II) dichloride (Sigma-Aldrich, St. Louis, MO) on Day 9 at 2 mg/kg in physiological saline.

### Preparation of Ascites-Derived Tumor Vaccines

To prepare vaccine using murine tumor (ascites) cells, peritoneal fluid was collected from mice with late stage BR5-Akt cancer. The intact peritoneal cavity was exposed and ascites, as well as two peritoneal wash samples with cold PBS, were collected using an 18-gauge needle and 5 mL syringe inserted in the hypogastric region and positioned towards the cecum. In addition, ascites fluid or peritoneal washing samples were collected from patients with a diagnosis of ovarian cancer at the time of surgical debulking in accordance with approved IRB Protocol #UNM INST 1509, entitled “Single Institution (UNM) Prospective Laboratory Study of Cancer and Immune Cells in the Ascites Fluid of Ovarian Cancer Patients to Test Alternative Therapies.” Human specimens were de-identified prior to transfer for research purposes. To isolate peritoneal cells, ascites were centrifuged at 1400 RPM for five minutes, after which the supernatant was removed, and RBCs were removed using ACK lysis buffer. Tumor cells were enriched using a 37 μm reversible strainer. EpCAM⁺ populations were evaluated by flow cytometry. Cells were then silicified and surface modified as previously described.

### Adoptive Transfer of CD8⁺ T Cells

Peritoneal washings were collected from tumor-bearing vaccinated (Day 33 post tumor challenge) and control (tumor and vaccine naïve) FVB mice as described above. Cells were enriched for CD8⁺ T cells using the negative selection mouse CD8a⁺ T cell isolation cell separation kit (Miltenyi Biotec, Bergisch Gladbach, Germany). Purification was confirmed post separation using flow cytometry. Cells were resuspended in cold PBS and adaptively transferred intraperitoneally (2×10⁵ cells/mouse) to tumor and vaccine naïve FVB mice. Control mice received sterile PBS IP. 24 hours after transfer of CD8⁺ T cells, recipient mice were challenged with intraperitoneal BR5-Akt-Luc2⁺ tumor cells (2×10⁵/mouse). Mice where then monitored for tumor progression using luminescence using an in vivo imaging system (IVIS SPECTRUM, PerkinElmer, Inc., Waltham, MA).

### Imaging Tumor Burden

For in vivo monitoring of tumor burden, mice with BR5-Akt-Luc2 tumors were administered 150 mg luciferin/kg by intraperitoneal injection, with a 10-minute delay before imaging. Mice were then anesthetized using 2.5% isoflurane, and 2D/3D bioluminescence images were acquired using an in vivo imaging system (XENOGEN IVIS SPECTRUM, PerkinElmer, Inc., Waltham, MA). ROI measurements of total flux (photons/sec) were acquired using LIVING IMAGE software (Perkin Elmer, Inc., Waltham, MA).

### Murine Tissue/Cell Collection

Mice were euthanized, spleens were mechanically dissociated, and RBC were eliminated using ACK or BD Pharm Lyse. Blood was collected by retro-orbital withdrawal using EDTA or heparin to prevent blood clotting. Omentum, peritoneal tumor, lungs, gut, brain, and kidneys were dissected out and fixed in 10% buffered formalin. Tissues were embedded in paraffin, sectioned, and stained with hematoxylin and eosin. Bright-field images were acquired using a dissection microscope equipped with a Sony CCD progressive scan color camera (World Precision Instruments, LLC, Sarasota, FL).

### Biodistribution of Vaccine

To track vaccine cells, BR5-Akt cancer cells were first incubated with Cy3-labeled MSN for 16 hours. Cells were then washed to remove free MSN and following silicification, 3×10⁶ Si-PEI-CpG-MPL cells in 200 μL PBS were administered intraperitoneally (IP) to FVB mice four days post IP tumor challenge. Twenty-four hours later, mice were euthanized, and peritoneal tissues were frozen in optimal cutting temperature (OCT) compound. Following sectioning, tissues were fixed in ice-cold acetone for 15 minutes, stained with anti-mouse CD11c antibody and mounted in Prolong Gold Mounting Media containing DAPI. Images were acquired using a 63×/1.4NA oil objective in sequential scanning mode using a confocal microscope (TCS SP8, Leica, Wetzlar, Germany).

### Immune Cell Phenotyping

Single-cell suspensions were first blocked with Fc receptor blockers (1 μg anti-CD16/CD32 (clone 2.4G2) and 1 μg mouse IgG. Next, samples were surface stained with conjugated primary antibodies (1:250 dilution) at room temperature for 30 minutes in the dark. Samples were then stained with LIVE/DEAD Fixable Aqua dead cell stain (Thermo Fisher Scientific, Inc., Waltham, MA) for 15 minutes at room temperature in the dark. For intracellular cytokine analysis, cells were stimulated using a cell stimulation cocktail (eBioscience, San Diego, CA; 500×) plus protein transport inhibitors cocktail (eBioscience, San Diego, CA; 500×) for four hours in RMPI complete media. Cell permeabilization for intracellular staining was done using the FoxP3/transcription factor staining buffer set (eBioscience, San Diego, CA). Phenotyping was performed on stained cells using a flow cytometer (ATTUNE NXT, Becton, Dickinson and Co., Franklin Lakes, NJ) and analyzed using FLOWJO (10.6) (FloJo Software, LLC, Ashland, OR).

### Cytokine Analysis

Neat peritoneal fluid from mice bearing BR5-Akt tumors at various stages of progression, with either no treatment or following treatment with dehydrated vaccine, was collected and stored at −80° C. Samples were evaluated using a custom MILLIPLEX MAP mouse high sensitivity T cell panel (EMD Millipore, MilliporeSigma, Burlington, MA) using the MAGPIX system (Luminex Corp., Austin, TX) without dilution as decribed by the vendor.

### Blood Metabolite Measurements

Anti-coagulated blood metabolites and complete blood counts were measured on Day 18 or 19 using the a comprehensive or partial diagnostic profile disc (V2, VETSCAN, Abaxis, Union City, CA) as described by the vendor.

### In Vitro Activation of Dendritic Cells by Combination Silicified Tumor Cells and Doxorubicin-Treated Cancer Cell Supernatant

Dendritic cells were seeded in 12-well plates at a density of 100,000 cells per well. After 24 hours, supernatant from cancer cells treated with doxorubicin (5 ng/mL or 50 ng/mL) was added, with or without 100,000 silicified tumor cells. Cells were labeled with fluorescent antibodies specific for CD40 and CD11c and analyzed using the Attune Flow Cytometer.

### Statistical Analysis

Measurements in this study were taked from distinct samples. Graphpad Prism was used to perform statistical analysis. Kaplan-Meier survival curves were analyzed using Log-rank Mantel-Cox and Match SPSS and SAS tests for two and multiple group comparisons, respectively. For tumor burden comparisons, multiple t-tests assuming all rows are sampled from populations with the same scatter and correction for multiple comparisons using the Holm-Sidak method were used. Column statistics were analyzed using unpaired, two-tailed parametric t tests with equal SD. Graphs include means and error bars, with the latter representing standard deviation.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text
SEQ ID NO: 1
SIINFEKL

1. A method comprising:
obtaining a dried silicified cell that has been stored for at least 24 hours without cryopreservation; and
rehydrating the dried silicified cell in a pharmaceutically acceptable carrier.

2. The method of claim 1, further comprising surface modifying the silicified cell with at least one immunogenic molecule.

3. The method of claim 1, further comprising administering the rehydrated silicified cell to a subject.

4. The method of claim 1, wherein the dried silicified cell has been stored for at least 14 days without cryopreservation.

5. The method of claim 1, wherein the silicified cell is a silicified tumor cell.

6. The method of claim 1, wherein the silicified cell is derived from a tumor cell line.

7. The method of claim 1, wherein the silicified cell is a de-differentiated cell.

8. A method of treating a tumor in a subject, the method comprising:
administering to the subject a tumor therapy effective to treat the tumor; and
administering to the subject a silicified cell vaccine effective to treat the tumor.

9. The method of claim 8, wherein the tumor therapy comprises a chemotherapeutic agent.

10. The method of claim 2, further comprising administering the rehydrated silicified cell to a subject.

11. The method of claim 2, wherein the silicified cell is a silicified tumor cell.

12. The method of claim 3, wherein the silicified cell is a silicified tumor cell.

13. The method of claim 10, wherein the silicified cell is a silicified tumor cell.

14. The method of claim 2, wherein the silicified cell is derived from a tumor cell line.

15. The method of claim 3, wherein the silicified cell is derived from a tumor cell line.

16. The method of claim 14, further comprising administering the rehydrated silicified cell to a subject.


SEQUENCE LISTING (1 sequence)

SEQ ID NO: 1 - protein, 8 aa, Artificial
  polypeptide
SIINFEKL